(19) United States
(12) Patent Application Publication
(10) Pub. No.: US 2026/0335227 A1
(43) Pub. Date: Oct. 29, 2026

(54) Implantable Device for Release of GLP-1 Receptor Agonists

(71) Applicant: Celanese EVA Performance Polymers LLC, Irving, TX (US)

(72) Inventors: Vijay Gyanani, Powell, OH (US); Jeffrey Haley, Norwood, OH (US)

(21) Appl. No.: 19/660,743

(22) Filed: Apr. 28, 2026

Related U.S. Application Data
(60) Provisional application No. 63/796,441, filed on Apr. 29, 2025.

(51) Int. Cl. A61K 9/00; A61K 9/14; A61K 38/26; A61K 47/26; A61K 47/32

(52) U.S. Cl. CPC A61K 9/0092; A61K 9/0024; A61K 9/145; A61K 9/146; A61K 38/26; A61K 47/26; A61K 47/32

(57) ABSTRACT

An implantable device for delivery of a therapeutic agent is provided. The device includes a core comprising a core polymer matrix within which is dispersed a therapeutic agent including one or more GLP-1 receptor agonists and at least one excipient. The core polymer matrix includes a ratio of one or more glucagon-like peptide-1 (GLP-1) receptor agonists to at least one excipient ranging from about 20:1 to about 1:20. The core polymer matrix also includes an ethylene vinyl acetate copolymer.

### RELATED APPLICATION

The present application is based upon and claims priority to U.S. Provisional Patent Application Ser. No. 63/796,441, having a filing date of Apr. 29, 2025, which is incorporated herein by reference.

### BACKGROUND OF THE INVENTION

Glucagon-like peptide-1 (“GLP-1”) is an endogenous incretin hormone produced by intestinal enteroendocrine L-cells following nutrient ingestion. GLP-1 produces many therapeutic effects by activating GLP-1 receptors distributed in the pancreas, gastrointestinal tract, brain, heart, and kidneys. For example, if blood glucose levels are elevated, GLP-1 promotes insulin secretion in the pancreas; however, it has minimal effect when blood glucose levels are normal. Because of this, GLP-1 receptor agonists can be used to treat high blood glucose levels associated with diabetes. Additionally, GLP-1 may decrease glucagon secretion, further reducing blood glucose levels. GLP-1 may also improve beta-cell function and inhibit beta-cell apoptosis, thereby slowing the progression of beta-cell failure in type 2 diabetes.

Treatment with GLP-1 receptor agonists can be long lasting and can require treatment for many weeks, months, or even years. Patients taking oral dosage forms may be required to take the oral dose at least one per day, if not multiple times per day, for the duration of treatment. Patients taking injectable dosage forms may have to administer multiple injections each month. Such continuous dosage over a long period of time can result in decreased patient compliance, as many patients either forget to take the medication or become tired of having to continually take the medication.

As such, a need continues to exist for treatment options that are capable of delivering one or more GLP-1 receptor agonists over a sustained period of time.

### SUMMARY OF THE INVENTION

In accordance with one embodiment of the present invention, an implantable device for delivery of a therapeutic agent, including a GLP-1 receptor agonist, is disclosed. The device includes a core comprising a core polymer matrix within which is dispersed a therapeutic agent including one or more GLP-1 receptor agonists and at least one excipient. The core polymer matrix includes a ratio of one or more glucagon-like peptide-1 (GLP-1) receptor agonists to at least one excipient ranging from about 20:1 to about 1:20. The core polymer matrix also includes an ethylene vinyl acetate copolymer.

Other features and aspects of the present invention are set forth in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended drawings in which:

FIG. 1 is a perspective view of one embodiment of the implantable device of the present invention;

FIG. 2 is a cross-sectional view of the implantable device of FIG. 1;

FIG. 3 is a perspective view of another embodiment of the implantable device of the present invention;

FIG. 4 is a cross-sectional view of the implantable device of FIG. 3; and

FIG. 5 illustrates gel and fibril formation of implant samples for Comparative Example 1.

FIG. 6 illustrates the cumulative release (mg) per day of liraglutide for Comparative Example 3.

FIG. 7 illustrates liraglutide release (mg/day) from implant samples for Example 25.

FIG. 8 illustrates liraglutide release (mg/day) from implant samples for Example 26.

FIG. 9 illustrates liraglutide cumulative release (mg) from implant samples for Example 25.

FIG. 10 illustrates liraglutide cumulative release (mg) from implant samples for Example 26.

FIG. 11 illustrates the percentage of liraglutide released from implant samples for Example 25.

FIG. 12 illustrates the percentage of liraglutide released from implant samples for Example 26.

Repeat use of references characters in the present specification and drawing is intended to represent same or analogous features or elements of the invention.

### DETAILED DESCRIPTION

It is to be understood by one of ordinary skill in the art that the present discussion is a description of exemplary embodiments only, and is not intended as limiting the broader aspects of the present invention.

As discovered by the inventors, release of GLP-1 receptor agonists from polymeric implants is unpredictable as some implants exhibit burst-like release and other implants do not provide substantial release. Further, many implants stopped releasing after a short period of time. Through control of the composition of the implant core and membrane as described herein, the inventors were able to produce a polymeric implant capable of releasing a GLP-1-receptor agonist for a prolonged period of time.

Generally speaking, the present invention is directed to an implantable device that is capable of delivering a GLP-1 receptor agonist for prohibiting and/or treating a condition, disease, and/or cosmetic state in a patient (e.g., human, pet, farm animal, racehorse, etc.). The condition and/or disease can include type-2 diabetes or any other disorders where a reduction in blood glucose levels is desirable. The implantable device includes a core comprising a core polymer matrix within which is dispersed a therapeutic agent including one or more GLP-1 receptor agonists and at least one excipient. The core polymer matrix includes a ratio of one or more glucagon-like peptide-1 (GLP-1) receptor agonists to at least one excipient ranging from about 20:1 to about 1:20. The core polymer matrix also includes a first ethylene vinyl acetate copolymer.

Various embodiments of the present invention will now be described in more detail.

### I. Core

As indicated above, the core polymer matrix contains at least a polymer that is generally hydrophobic in nature so that it can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the core polymer matrix. Typically, the melt flow index of the hydrophobic polymer ranges from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-13 at a temperature of 190 °C and a load of 2.16 kilograms.

In certain embodiments, the core polymer matrix may contain a semi-crystalline olefin copolymer. The melting temperature of such an olefin copolymer may, for instance, range from about 40 °C to about 140 °C, in some embodiments from about 50 °C to about 125 °C, and in some embodiments, from about 60 °C to about 120 °C, as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene(meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene(meth)acrylate polymers (e.g., ethylene methylacrylate copolymers, ethylene ethyl acrylate copolymers, ethylene butyl acrylate copolymers, etc.), and so forth. Regardless of the particular monomers selected, certain aspects of the copolymer can be selectively controlled to help achieve the desired release properties. For instance, the polar monomeric content of the copolymer may be selectively controlled to be within a range of from about 10 wt. % to about 60 wt. %, in some embodiments, from about 20 wt. % to about 60 wt. %, and in some embodiments, from about 25 wt. % to about 50 wt. %. Conversely, the olefin monomeric content of the copolymer may be likewise within a range of from about 40 wt. % to about 90 wt. %, in some embodiments, from about 40 wt. % to about 80 wt. %, and in some embodiments, from about 50 wt. % to about 75 wt. %.

In one particular embodiment, for example, the core polymer matrix may contain at least one ethylene vinyl acetate polymer, which is a copolymer that is derived from at least one ethylene monomer and at least one vinyl acetate monomer. In certain cases, the present inventors have discovered that certain aspects of the copolymer can be selectively controlled to help achieve the desired release properties. For instance, the vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments from about 1 to about 200 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190 °C and a load of 2.16 kilograms. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55). In embodiments, the ethylene vinyl acetate copolymer in the core polymer matrix is from about 20 wt. % to about 90 wt. %, such as from about 30 wt. % to about 80 wt. %, such as from about 40 wt. % to about 70 wt. %.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain embodiments, it may also be desirable to employ blends of an ethylene vinyl acetate copolymer and another hydrophobic polymer such that the overall blend and polymer matrix have a melting temperature and/or melt flow index within the range noted above. For example, the polymer matrix may contain a first ethylene vinyl acetate copolymer and a second ethylene vinyl acetate copolymer having a melting temperature that is greater than the melting temperature of the first copolymer. The second copolymer may likewise have a melt flow index that is the same, lower, or higher than the corresponding melt flow index of the first copolymer. The first copolymer may, for instance, have a melting temperature of from about 20 °C to about 60 °C, in some embodiments from about 25 °C to about 55 °C, and in some embodiments, from about 30 °C to about 50 °C, such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190 °C and a load of 2.16 kilograms. The second copolymer may likewise have a melting temperature of from about 50 °C to about 100 °C, in some embodiments from about 55 °C to about 90 °C, and in some embodiments, from about 60 °C to about 80 °C, such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190 °C and a load of 2.16 kilograms. The first copolymer may constitute from about 20 wt. % to about 80 wt. %, in some embodiments from about 30 wt. % to about 70 wt. %, and in some embodiments, from about 40 wt. % to about 60 wt. % of the polymer matrix, and the second copolymer may likewise constitute from about 20 wt. % to about 80 wt. %, in some embodiments from about 30 wt. % to about 70 wt. %, and in some embodiments, from about 40 wt. % to about 60 wt. % of the polymer matrix.

In certain cases, ethylene vinyl acetate copolymer(s) constitute the entire polymer content of the core polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix.

One or more therapeutic agents (e.g., GLP-1 receptor agonists) are also dispersed within the core polymer matrix that are capable of prohibiting and/or treating a condition, disease, and/or cosmetic state in a patient. The therapeutic agent may be prophylactically, therapeutically, and/or cosmetically active, systemically, or locally. The therapeutic agent can be homogenously dispersed within the core polymer matrix. Typically, therapeutic agents will constitute from about 5 wt. % to about 60 wt. %, in some embodiments from about 10 wt. % to about 50 wt. %, and in some embodiments, from about 15 wt. % to about 45 wt. % of the core. In certain embodiments, therapeutic agents will constitute from about 20 wt. % to about 80 wt. % of the core, and in some embodiments, from about 30 wt. % to about 70 wt. % of the core.

The therapeutic agent can be either naturally occurring or man-made by any method known in the art. Typically, it is also desired that the therapeutic agent is stable at high temperatures so that it can be incorporated into the polymer matrix at or near the melting temperature of the hydrophobic polymer employed in the core polymer matrix. For example, the therapeutic agent typically remains stable at temperatures of from about 25 °C to about 120 °C, in some embodiments from about 40 °C to about 110 °C, in some embodiments from about 40 °C to about 100 °C, in some embodiments from about 40 °C to about 80 °C, and in some embodiments, from about 50 °C to about 70 °C. Suitable therapeutic agents will be further discussed hereinbelow.

At least one excipient is also dispersed within the core polymer matrix. The at least one excipient can include a mixture of different excipients. The excipient can include non-ionic surfactants and derivatives thereof, cationic surfactants and derivatives thereof, anionic surfactants and derivatives thereof, sugars or sugar derivatives, one or more glycols or polyglycols (e.g., polyethylene glycol) and derivatives thereof, one or more polymers, or combinations and mixtures thereof.

The excipient may be homogenously dispersed within the core polymer matrix. The excipient can be selected so as to prevent gelling of the therapeutic agent prior to forming the implant, which can facilitate a higher loading percentage of the therapeutic agent. The excipient typically constitutes from about 0.25 wt. % to about 80 wt. %, in some embodiments from about 1 wt. % to about 70 wt. %, and in some embodiments, from about 5 wt. % to about 45 wt. % of the core of the core.

In some embodiments, a combination of the therapeutic agent and the excipient will constitute from about 5 wt. % to about 95 wt. %, and in some embodiments, from about 10 wt. % to about 80 wt. %, and in some embodiments, from about 30 wt. % to about 75 wt. % of the core, while the core polymer matrix constitutes from about 5 wt. % to about 60 wt. %, and in some embodiments, from about 20 wt. % to about 65 wt. %, and in some embodiments, from about 25 wt. % to about 60 wt. % of the core. In some embodiments, the weight ratio of the therapeutic agent to the excipient ranges from about 20:1 to about 1:20, and in some embodiments, from about 10:1 to about 1:10, and in some embodiments, from about 1:5 to about 5:1.

To help further control the release rate from the implantable device, a hydrophilic compound may also be incorporated into the core that is soluble and/or swellable in water. When employed, the weight ratio of the ethylene vinyl acetate copolymer(s) the hydrophilic compounds within the core may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Optionally, the core can include a plurality of water-soluble particles distributed within the core polymer matrix. The particle size of the water-soluble particles is controlled to help achieve the desired delivery rate. More particularly, the median diameter (D50) of the particles is about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles are also selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt. % to about 50 wt. %, in some embodiments from about 2 wt. % to about 45 wt. %, in some embodiments from about 4 wt. % to about 40 wt. %, and in some embodiments, from about 5 wt. % to about 30 wt. % of the core.

Regardless of the particular components employed, the core may be formed through a variety of known techniques, such as by hot-melt extrusion, injection molding, solvent casting, dip coating, spray coating, microextrusion, coacervation, compression molding (e.g., vacuum compression molding), etc. In one embodiment, a hot-melt extrusion technique may be employed. Hot-melt extrusion is generally a solvent-free process in which the components of the core (e.g., hydrophobic polymer, therapeutic agent(s), optional excipients, etc.) may be melt blended and optionally shaped in a continuous manufacturing process to enable consistent output quality at high throughput rates. This technique is particularly well suited to various types of hydrophobic polymers, such as olefin copolymers. Namely, such copolymers typically exhibit a relatively high degree of long-chain branching with a broad molecular weight distribution. This combination of traits can lead to shear thinning of the copolymer during the extrusion process, which help facilitates hot-melt extrusion. Furthermore, the polar comonomer units (e.g., vinyl acetate) can serve as an “internal” plasticizer by inhibiting crystallization of the polyethylene chain segments. This may lead to a lower melting point of the olefin copolymer, which improves the overall flexibility of the resulting material and enhances its ability to be formed into devices of a wide variety of shapes and sizes.

During a hot-melt extrusion process, melt blending may occur at a temperature range of from about 20 °C to about 200 °C, in some embodiments, from about 30 °C to about 150 °C, in some embodiments from about 40 °C to about 100 °C, and in some embodiments, in some embodiments from about 100 °C to about 120 °C, to form a polymer composition. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical and it may contain any number and/or orientation of threads and channels as is known in the art. For example, the screw typically contains a thread that forms a generally helical channel radially extending around a core of the screw. A feed section and melt section may be defined along the length of the screw. The feed section is the input portion of the barrel where the olefin copolymer(s) and/or therapeutic agent(s) are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Once melt blended together, the resulting polymer composition may be in the form of pellets, sheets, fibers, filaments, etc., which may be shaped into the core using a variety of known shaping techniques, such as injection molding, compression molding, nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present disclosure. In one embodiment, an injection molding apparatus may be employed that includes a first mold base and a second mold base, which together define a mold cavity having the shape of the core. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape of the core (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 30 °C to about 120 °C, in some embodiments from about 60 °C to about 110 °C, and in some embodiments, from about 30 °C to about 60 °C.

As indicated above, another suitable technique for forming a core of the desired shape and size is three-dimensional printing. During this process, the polymer composition may be incorporated into a printer cartridge that is readily adapted for use with a printer system. The printer cartridge may, for example, contains a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present disclosure. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50 °C to about 120 °C, in some embodiments from about 60 °C to about 110 °C, and in some embodiments, from about 70 °C to about 90 °C. A vacuum source may also apply a negative pressure to the precursor during molding to help ensure that it retains a precise shape. Examples of such compression molding techniques are described, for instance, in U.S. Pat. No. 10,625,444 to Treffer, et al., which is incorporated herein in its entirety by reference thereto.

Prior to forming the implant according to the methods disclosed, the therapeutic agent can be mixed with one or more suitable excipients. For example, one or more therapeutic agents can be mixed with an excipient in a suitable solvent. As discovered by the inventors of the present disclosure, when one or more therapeutic agents are formulated prior to mixing with the core polymer matrix, GLP-1 receptor agonists tend to form gel-like consistencies, which severely reduce the loading amount of the GLP-1 receptor agonist, causing waste of valuable materials. However, when the GLP-1 receptor agonists are first formulated in a mixture including the one or more excipients and a suitable solvent in the amounts disclosed herein, more GLP-1 receptor agonists remain un-gelled in the solution facilitating higher drug loading when combined with the core polymer matrix to make the implantable device.

The method of preparing the implant can include forming a drug mixture prior to forming the implant. The drug mixture includes at least one therapeutic agent, at least one excipient, and at least one solvent. Further, in some embodiments, the concentration of therapeutic agent in the drug mixture ranges from about 0.001 mg/mL to about 300 mg/mL, such as from about 1 mg/mL to about 200 mg/mL, such as from about 10 mg/mL to about 150 mg/mL. Suitable solvents can include aqueous solvents (e.g., water) or organic solvents. Examples of such solvents may be phosphate buffered saline, N-methyl-2-pyrrolidone, propylene glycol, triethylene glycol, tetraethylene glycol, etc., and other solvents described herein.

After forming the drug mixture containing one or more therapeutic agents, at least one excipient, and the solvent, the drug mixture can be dried according to any suitable drying procedure to remove solvent from the drug mixture forming a dried drug mixture. Suitable drying methods include vacuum drying, lyophilization, oven drying, etc. The weight ratio of the one or more therapeutic agents to the excipient in the dried drug mixture can range from about 20:1 to about 1:20.

After drying, the dried drug mixture can then be mixed with the core polymer matrix material via techniques disclosed herein, such as hot-melt extrusion, injection molding, solvent casting, dip coating, spray coating, microextrusion, coacervation, compression molding (e.g., vacuum compression molding), etc. to form the implantable device.

### II. Therapeutic Agent

One or more therapeutic agents are also dispersed within the core polymer matrix that are capable of prohibiting and/or treating a condition, disease, and/or cosmetic state a patient. The therapeutic agent may be prophylactically, therapeutically, and/or cosmetically active, systemically or locally. The therapeutic agent can include one or more GLP-1 receptor agonists. GLP-1 receptor agonists include dulaglutide, exenatide, semaglutide, liraglutide, lixisenatide, and tirzepatide. Combinations of GLP-1 receptor agonists can also be included in the core.

Dulaglutide is a human GLP-1 receptor agonist. The molecule is a fusion protein that includes 2 identical, disulfide-linked chains, each containing an N-terminal GLP-1 analog sequence covalently linked to the Fc portion of a modified human immunoglobulin G4 (IgG4) heavy chain by a small peptide linker. The GLP-1 analog portion of dulaglutide is 90% homologous to native human GLP-1 (7-37). Structural modifications were introduced in the GLP-1 part of the molecule responsible for interaction with the enzyme dipeptidyl-peptidase-IV (DPP-4). Additional modifications were made in an area with a potential T-cell epitope and in the areas of the IgG4 Fc part of the molecule responsible for binding the high-affinity Fc receptors and half-antibody formation. The overall molecular weight of dulaglutide is approximately 63 kilodaltons.

Dulaglutide activates the GLP-1 receptor, a membrane-bound cell-surface receptor coupled to adenylyl cyclase in pancreatic beta cells. Dulaglutide increases intracellular cyclic AMP (CAMP) in beta cells, leading to glucose-dependent insulin release. Dulaglutide also decreases glucagon secretion and slows gastric emptying.

Exenatide is a synthetic peptide that was originally identified in the lizard Heloderma suspectum. Exenatide is a 39-amino acid peptide amide having the empirical formula C₁₈₄H₂₈₂N₅₀O₆₀S and molecular weight of 4186.6 Daltons. The amino acid sequence of exenatide partially overlaps that of human GLP-1. Exenatide has been shown to bind and activate the human GLP-1 receptor in vitro. Such binding leads to an increase in both glucose-dependent synthesis of insulin and in vivo secretion of insulin from pancreatic beta cells, by mechanisms involving cyclic AMP and/or other intracellular signaling pathways. Exenatide promotes insulin release from pancreatic beta cells in the presence of elevated glucose concentrations

Semaglutide is a human GLP-1 receptor agonist. The main protraction mechanism of semaglutide is albumin binding, facilitated by modification of position 26 lysine with a hydrophilic spacer and a C18 fatty di-acid. Furthermore, semaglutide is modified in position 8 to provide stabilization against degradation by DPP-4. A minor modification is also made in position 34 to ensure the attachment of only one fatty di-acid. The molecular formula of semaglutide is C₁₈₇H₂₉₁N₄₅O₅₉ and the molecular weight is 4113.58 g/mol.

Liraglutide is an analog of human GLP-1 and acts as a GLP-1 receptor agonist. The peptide precursor of liraglutide has been engineered to be 97% homologous to native human GLP-1 by substituting arginine for lysine at position 34. Liraglutide is made by attaching a C-16 fatty acid (palmitic acid) with a glutamic acid spacer on the remaining lysine residue at position 26 of the peptide precursor. The molecular formula of liraglutide is C₁₇₂H₂₆₅N₄₃O₅₁ and the molecular weight is 3751.2 Daltons.

Lixisenatide is modified from exendin-4 and is a 44-amino-acid peptide with the C-terminal Pro replaced by 6 Lys residues. Its molecular weight is 4858.5 and its molecular formula is C₂₁₅H₃₄₇N₆₁O₆₅S. In vitro studies found that lixisenatide has four times greater affinity for the GLP-1 receptor than native GLP-1 and exenatide.

Tirzepatide is a dual agonist of the glucose-dependent insulinotropic polypeptide (GIP) receptor and the glucagon-like peptide-1 (GLP-1) receptor. The peptide backbone of tirzepatide is a 39-amino acid sequence engineered to be based on the native GIP peptide, with selective modifications introduced to confer balanced agonist activity at both the GIP and GLP-1 receptors. Tirzepatide is made by attaching a C-20 fatty diacid moiety via a hydrophilic linker consisting of two mini-PEG (polyethylene glycol) spacers and a gamma-glutamic acid unit to a lysine residue at position 20 of the peptide backbone. The molecular formula of tirzepatide is C₂₂₅H₃₄₈N₄₈O₆₈ and the molecular weight is approximately 4,813.5 Daltons.

In certain embodiments, the therapeutic agent can include one or more GLP-1 receptor agonists having a molecular weight of about 5,000 Da or less, in some embodiments from about 1000 Da to about 5000 Da, in some embodiments from about 2500 Da to about 5000 Da, and in some embodiments from about 3500 Da to about 5000 Da, or any range therebetween.

In certain other embodiments, the GLP-1 receptor agonist can include molecules that are selective for the human GLP-1 receptor. For instance, the GLP-1 receptor agonist can include molecules, compounds, metabolites, and/or isomers that are selective for the GLP-1 receptor. For instance, in certain embodiments the GLP-1 receptor agonist can include small molecules or peptide fragments. Additionally, in certain embodiments native GLP-1 can be utilized in the implantable device disclosed herein. In such embodiments, the native GLP-1 are formulated with one or more excipients in order to improve the extrusion process of the implant.

### III. Excipient

At least one excipient is dispersed within the core polymer matrix. The excipient may be employed prior to forming the implant to increase the amount of therapeutic agent loaded into the implant. The excipient may be non-ionic surfactants, anionic surfactants, a cationic surfactants, sugars or sugar derivatives, small lipophilic molecules and derivatives thereof, glycols, polymers, and combinations and mixtures thereof.

In some instances, a non-ionic surfactant may be employed prior to forming the implant to improve therapeutic agent loading in the implant. Non-ionic surfactants are surfactants that are not electrically charged and do not ionize. Examples of such include, for instance, polyoxyethylene laurel ether, polyoxyl 40 hydrogenated castor oil, diethanolamine, polyethylene glycol 660 12-hydoxystearate, D-alpha-tocopherol polyethylene glycol 1000 succinate, Polysorbate 80, Gelucire 59/14, phenol, N-Decanoyl-N-methylglucamine, caprylocaproyl polyoxyl-8 glycerides, glycol-diosgenin, derivatives thereof, etc., and combinations and mixtures thereof.

In some instances, a cationic surfactant may be employed prior to forming the implant to improve therapeutic agent loading in the implant. Cationic surfactants are surfactants that are positively charged. Cationic surfactants may also be employed due to their antimicrobial properties. Examples of such include, for instance, benzalkonium chloride, derivatives thereof, etc., and combinations and mixtures thereof.

In some instances, an anionic surfactant may be employed prior to forming the implant to improve therapeutic agent loading in the implant. Anionic surfactants are surfactants that are negatively charged. Examples of such include, for instance, sodium lauryl sulfate, triethanolamine, derivatives thereof, etc.

In some instances, a sugar or sugar derivative may be employed prior to forming the implant to improve therapeutic agent loading in the implant. Examples of such include, for instance, glycerol, mannitol, glucose, beta cyclodextrin, hydroxypropyl beta cyclodextrin, propylene glycol, derivatives thereof, etc.

In some instances, a polymer may be employed to help the therapeutic agent remain suspended in the core. Examples of such include, for instance, hydroxylpropyl methyl cellulose, Kollidon 30, derivatives thereof, etc.

### IV. Membrane Layer(s)

As indicated above, the implantable device can optionally include one or more membrane layers (e.g., a first membrane layer) that is positioned adjacent to an outer surface of a core. Additional membrane layers (e.g., a second membrane layer, a third membrane layer, etc.) may be layered on the core as desired. The number of membrane layers may vary depending on the particular configuration of the device, the nature of the therapeutic agent, and the desired release profile.

Regardless of the particular configuration employed, the membrane polymer matrix contains at least one ethylene vinyl acetate copolymer, such as described in more detail above. The vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190 °C and a load of 2.16 kilograms. The melting temperature of the ethylene vinyl acetate copolymer may also range from about 40 °C to about 140 °C, in some embodiments from about 50 °C to about 125 °C, and in some embodiments, from about 60 °C to about 120 °C, as determined in accordance with ASTM D3418-15. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVAR 4030AC); Dow under the designation ELVAX® (e.g., ELVAX® 40W); and Arkema under the designation EVATANE® (e.g., EVATANE 40-55). In embodiments, the ethylene vinyl acetate copolymer in the membrane polymer matrix is from about 20 wt. % to about 90 wt. %, such as from about 30 wt. % to about 80 wt. %, such as from about 40 wt. % to about 70 wt. %.

In certain cases, ethylene vinyl acetate copolymer(s) constitute the entire polymer content of the membrane polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix. The membrane polymer matrix typically constitutes from about 50 wt. % to 99 wt. %, in some embodiments, from about 55 wt. % to about 98 wt. %, in some embodiments, from about 60 wt. % to about 96 wt. %, and in some embodiments, from about 70 wt. % to about 95 wt. % of a membrane layer.

To help further control the release rate from the implantable medical device, a hydrophilic compound may also be incorporated into the membrane layer(s) that is soluble and/or swellable in water. When employed, the weight ratio of the ethylene vinyl acetate copolymer(s) the hydrophilic compounds within the membrane layer may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Optionally, the membrane layer(s) can include a plurality of water-soluble particles distributed within a membrane polymer matrix. The particle size of the water-soluble particles is controlled to help achieve the desired delivery rate. More particularly, the median diameter (D50) of the particles is about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles are also selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt. % to about 50 wt. %, in some embodiments from about 2 wt. % to about 45 wt. %, in some embodiments from about 4 wt. % to about 40 wt. %, and in some embodiments, from about 5 wt. % to about 30 wt. % of a membrane layer.

For instance, optionally, the membrane layer(s) can include mannitol distributed within a membrane polymer matrix. If utilized, mannitol particles present in the membrane layer(s) may have an average particle size of at least about 25 μm and less than about 125 μm. In some embodiments, if utilized, mannitol particles present in the membrane layer(s) may have an average particle size of from about 25 μm to about 125 μm.

When employing multiple membrane layers, it is typically desired that each membrane layer contains a polymer matrix includes an ethylene vinyl acetate copolymer. Additionally, each of the membrane layers can include a plurality of water-soluble particles distributed within a membrane polymer matrix that includes an ethylene vinyl acetate copolymer. For example, a first membrane layer may contain first water-soluble particles distributed within a first membrane polymer matrix and a second membrane layer may contain second water-soluble particles distributed within a second membrane polymer matrix. In such embodiments, the first and second polymer matrices may each contain an ethylene vinyl acetate copolymer. The water-soluble particles and ethylene vinyl acetate copolymer(s) within one membrane layer may be the same or different than those employed in another membrane layer. In one embodiment, for instance, both the first and second membrane polymer matrices employ the same ethylene vinyl acetate copolymer(s) and the water-soluble particles within each layer have the same particle size and/or are formed from the same material. Likewise, the ethylene vinyl acetate copolymer(s) used in the membrane layer(s) may also be the same or different from the hydrophobic polymer(s) employed in the core. In one embodiment, for instance, both the core and the membrane layer(s) employ the same ethylene vinyl acetate copolymer. In yet other embodiments, the membrane layer(s) may employ an ethylene vinyl acetate copolymer that has a lower melt flow index than a hydrophobic polymer employed in the core. Among other things, this can further help control the release of the therapeutic agent from the device. For example, the ratio of the melt flow index of a hydrophobic polymer employed in the core to the melt flow index of an ethylene vinyl acetate copolymer employed in the membrane layer(s) may be from about 1 to about 20, in some embodiments, about 2 to about 15, and in some embodiments, from about 4 to about 12.

If desired, membrane layer(s) used in the device may optionally contain a therapeutic agent, such as described above, which is also dispersed within the membrane polymer matrix. The therapeutic agent in the membrane layer(s) may be the same or different than the therapeutic agent employed in the core. When such a therapeutic agent is employed in a membrane layer, the membrane layer generally contains the therapeutic agent in an amount such that the ratio of the concentration (wt. %) of the therapeutic agent in the core to the concentration (wt. %) of the therapeutic agent in the membrane layer is greater than 1, in some embodiments about 1.5 or more, and in some embodiments, from about 1.8 to about 4. When employed, therapeutic agents typically constitute only from about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of therapeutic agents prior to release from the core. When multiple membrane layers are employed, each membrane layer may generally contain the therapeutic agent in an amount such that the ratio of the weight percentage of the therapeutic agent in the core to the weight percentage of the therapeutic agent in the membrane layer is greater than 1, in some embodiments about 1.5 or more, and in some embodiments, from about 1.8 to about 4.

The membrane layer(s) may also optionally contain one or more excipients as described above, such as radiocontrast agents, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

One or more nonionic, anionic, and/or amphoteric surfactants may also be employed to help create a uniform dispersion. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of the core or membrane layers. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted or unsubstituted, and may contain from 6 to 22 carbon atoms, in some embodiments from 8 to 18 carbon atoms, and in some embodiments, from 12 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1,4-sorbitan ester. The addition of polyoxyethylene converts the lipophilic sorbitan ester surfactant to a hydrophilic surfactant that is generally soluble or dispersible in water. Such materials are commercially available under the designation TWEEN® (e.g., TWEEN® 80, or polyethylene (20) sorbitan monooleate).

The membrane layer(s) may be formed using the same or a different technique than used to form the core, such as by hot-melt extrusion, compression molding (e.g., vacuum compression molding), injection molding, solvent casting, dip coating, spray coating, microextrusion, coacervation, etc. In one embodiment, a hot-melt extrusion technique may be employed. The core and membrane layer(s) may also be formed separately or simultaneously. In one embodiment, for instance, the core and membrane layer(s) are separately formed and then combined together using a known bonding technique, such as by stamping, hot sealing, adhesive bonding, etc. Compression molding (e.g., vacuum compression molding) may also be employed to form the implantable device. As described above, the core and membrane layer(s) may be each individually formed by heating and compressing the respective polymer compression into the desired shape while under vacuum. Once formed, the core and membrane layer(s) may be stacked together to form a multi-layer precursor and thereafter and compression molded in the manner as described above to form the resulting implantable device.

As indicated above, the implantable device can include at least one membrane layer that is positioned adjacent to an outer surface of a core. The number of membrane layers may vary depending on the particular configuration of the device, the nature of the therapeutic agent, and the desired release profile. For example, the device may contain only one membrane layer.

Regardless of the particular configuration employed, the membrane layer(s) generally contain a membrane polymer matrix that contains a hydrophobic polymer, such as described above. The polymer matrix typically constitutes from about 30 wt. % to 100 wt. %, in some embodiments, from about 40 wt. % to about 99 wt. %, and in some embodiments, from about 50 wt. % to about 90 wt. % of a membrane layer. When employing multiple membrane layers, it is typically desired that each membrane layer contains a polymer matrix that includes such a hydrophobic polymer. For example, a first membrane layer may contain a first polymer matrix and a second membrane layer may contain a second polymer matrix. In such embodiments, the first and second polymer matrices each contain a hydrophobic polymer, which may be the same or different. Likewise, the hydrophobic polymer used in the membrane layer may also be the same or different from the hydrophobic polymer employed in the core. In one embodiment, for instance, both the core and the membrane layer(s) employ the same hydrophobic polymer (e.g., α-olefin copolymer). In yet other embodiments, the membrane layer(s) may employ a hydrophobic polymer (e.g., α-olefin copolymer) that has a lower melt flow index than a polymer employed in the core. Among other things, this can further help control the release of the therapeutic agent from the device. For example, the ratio of the melt flow index of a hydrophobic polymer employed in the core to the melt flow index of a hydrophobic polymer employed in the membrane layer(s) may be from about 1 to about 20, in some embodiments, about 2 to about 15, and in some embodiments, from about 4 to about 12. The melt flow index of the hydrophobic polymer in the membrane layer(s) may, for example, range from about 1 to about 80 g/10 min, in some embodiments from about 2 to about 70 g/10 min, and in some embodiments, from about 5 to about 60 g/10 min, as determined in accordance with ASTM D1238-13 at a temperature of 190 °C and a load of 2.16 kilograms. Examples of suitable ethylene vinyl acetate copolymers that may be employed include those available from Celanese under the designation ATEVAR (e.g., ATEVAR 4030AC or 2861A).

As indicated above, the membrane layer(s) used in the device may optionally contain a therapeutic agent (e.g., a GLP-1 receptor agonist), such as described above, which is dispersed within the polymer matrix. The therapeutic agent in the membrane layer(s) may be the same or different than the therapeutic agent employed in the core. Regardless, when such a therapeutic agent is employed in a membrane layer, the membrane layer generally contains the therapeutic agent in an amount such that the ratio of the concentration (wt. %) of the therapeutic agent in the core to the concentration (wt. %) of the therapeutic agent in the membrane layer is greater than 1, in some embodiments about 1.5 or more, and in some embodiments, from about 1.8 to about 4. When employed, therapeutic agents typically constitute only from about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of such therapeutic agents prior to release from the core. When multiple membrane layers are employed, each membrane layer may generally contain the therapeutic agent in an amount such that the ratio of the weight percentage of the therapeutic agent in the core to the weight percentage of the therapeutic agent in the membrane layer is greater than 1, in some embodiments about 1.5 or more, and in some embodiments, from about 1.8 to about 4.

The membrane layer(s) and/or the core may also optionally contain one or more excipients as described above, such as radiocontrast agents, hydrophilic compounds, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

To help further control the release rate from the implantable device, for example, a hydrophilic compound may also be incorporated into the polymer matrix of the membrane layer(s) that is soluble and/or swellable in water. When employed, the weight ratio of the hydrophobic polymers to the hydrophilic compounds within the membrane polymer matrix may range from about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 50 wt. %, in some embodiments from about 2 wt. % to about 40 wt. %, and in some embodiments, from about 5 wt. % to about 30 wt. % of the membrane polymer matrix, while hydrophobic polymers typically constitute from about 50 wt. % to about 99 wt. %, in some embodiments from about 60 wt. % to about 98 wt. %, and in some embodiments, from about 70 wt. % to about 95 wt. % of the membrane polymer matrix. In such embodiments, hydrophilic compounds may likewise constitute from about 1 wt. % to about 50 wt. %, in some embodiments from about 2 wt. % to about 40 wt. %, and in some embodiments, from about 5 wt. % to about 30 wt. % of a membrane layer. Suitable hydrophilic compounds may include, for instance, polymers, non-polymeric materials (e.g., glycerin, sugars, salts, peptides, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

The membrane layer(s) may be formed using the same or a different technique than used to form the core, such as by hot-melt extrusion, injection molding, solvent casting, dip coating, spray coating, microextrusion, coacervation, etc. In one embodiment, a hot-melt extrusion technique may be employed. The core and membrane layer(s) may also be formed separately or simultaneously. In one embodiment, for instance, the core and membrane layer(s) are separately formed and then combined together using a known bonding technique, such as by stamping, hot sealing, adhesive bonding, etc.

### V. Use of Device

The implantable device of the present invention may be used in a variety of different ways to treat prohibit and/or treat a condition, disease, or cosmetic state in a patient. The device may be implanted subcutaneously, orally, rectally, mucosally, etc., using standard techniques. The delivery route may be intrapulmonary, gastroenteral, subcutaneous, intramuscular, or for introduction into the central nervous system, intraperitoneum or for intraorgan delivery. The term “implantable device” as used herein is intended to cover a variety of implantable or insertable devices and associated methods of use. For example, the implantable device can be inserted subcutaneously using standard techniques. In such embodiments, the delivery route may be subcutaneous.

As noted above, the implantable device may be particularly suitable for delivering a GLP-1 receptor agonist for the treatment of diabetes (e.g., type 2 diabetes). The device may also be employed together with other therapies for the treatment of diabetes, including insulin injections and/or metformin administration. In such embodiments, these additional therapeutic agents can be administered to the patient in a variety of dosage forms, including, oral dosage forms, intravenous dosage forms, subcutaneous dosage forms, including depot injections, hydrogel injections, intramuscular injections, etc., or intravaginal dosage forms. Additional therapeutic agents can be administered via any suitable route and can be used in combination with the implantable device disclosed herein. The implantable device may also be particularly suitable for treating metabolic syndrome, atherosclerosis, dyslipidemia, obesity, and/or adipositas.

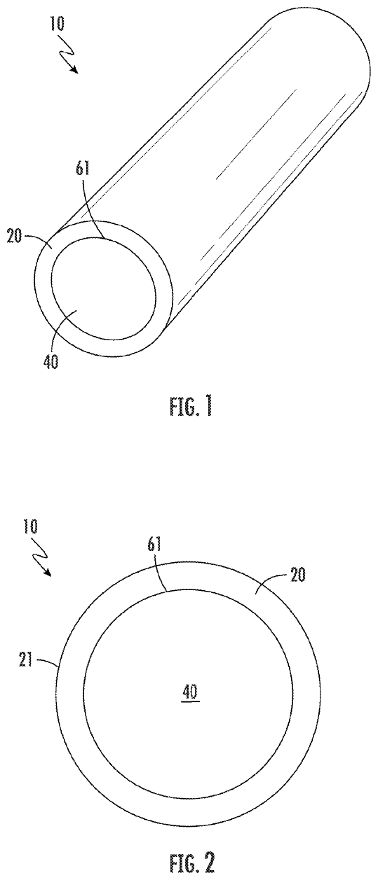

Referring to FIGS. 1-2, for example, one embodiment of an implantable device 10 is shown that contains a core 40 having a generally circular cross-sectional shape and is elongated so that the resulting device is generally cylindrical in nature. The core 40 defines an outer circumferential surface 61 about which a membrane layer 20 is circumferentially disposed. Similar to the core 40, the membrane layer 20 also has a generally circular cross-sectional shape and is elongated so that it covers the entire length of the core 40. During use of the device 10, a therapeutic agent is capable of being released from the core 40 and through the membrane layer 20 so that it exits from an external surface 21 of the device. While a device 10 having a core 40 and membrane layer 20 is depicted, it should be appreciated that the membrane layer 20 is optional and the implant 10 can be a monolithic implant containing only the core 40.

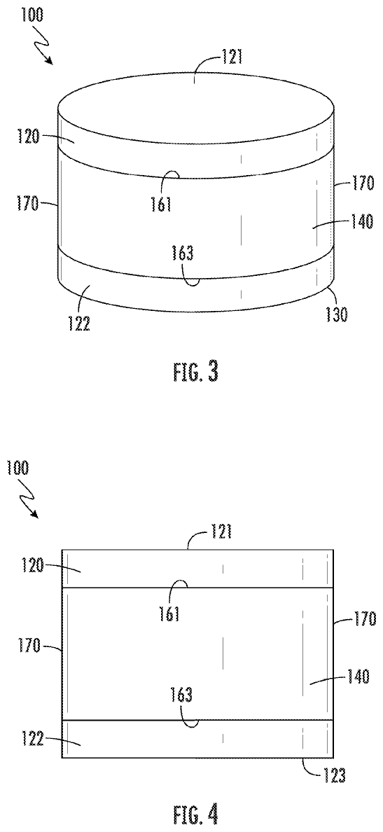

Of course, in other embodiments, the device may contain multiple membrane layers. In the device of FIGS. 1-2, for example, one or more additional membrane layers (not shown) may be disposed over the membrane layer 20 to help further control release of the therapeutic agent. In other embodiments, the device may be configured so that the core is positioned or sandwiched between separate membrane layers. Referring to FIGS. 3-4, for example, one embodiment of an implantable device 100 is shown that contains a core 140 having a generally circular cross-sectional shape and is elongated so that the resulting device is generally disc-shaped in nature. The core 140 defines an upper outer surface 161 on which is positioned a first membrane layer 120 and a lower outer surface 163 on which is positioned a second membrane layer 122. Similar to the core 140, the first membrane layer 120 and the second membrane layer 122 also have a generally circular cross-sectional shape that generally covers the core 140. If desired, edges of the membrane layers 120 and 122 may also extend beyond the periphery of the core 140 so that they can be sealed together to cover any exposed areas of an external circumferential surface 170 of the core 140. During use of the device 100, a therapeutic agent is capable of being released from the core 140 and through the first membrane layer 120 and second membrane layer 122 so that it exits from external surfaces 121 and 123 of the device. Of course, if desired, one or more additional membrane layers (not shown) may also be disposed over the first membrane layer 120 and/or second membrane layer 122 to help further control release of the therapeutic agent.

The implantable device may have a variety of different geometric shapes, such as cylindrical (rod), disc, ring, doughnut, helical, elliptical, triangular, ovular, etc. In one embodiment, for example, the device may have a generally circular cross-sectional shape so that the overall structure is in the form of a cylinder (rod) or disc. In such embodiments, the device will typically have a diameter of from about 0.5 to about 50 millimeters, in some embodiments from about 1 to about 40 millimeters, and in some embodiments, from about 5 to about 30 millimeters. The length of the device may vary, but is typically in the range of from about 1 to about 60 millimeters, in some embodiments from about 10 to about 40 millimeters. Cylindrical devices may, for instance, have a length of from about 5 to about 50 millimeters, while disc-shaped devices may have a length of from about 0.5 to about 5 millimeters. Certain disc-shaped devices may have a thickness ranging from about 1 mm to about 5 mm, such as from about 2 mm to about 4 mm, such as about 3 mm, and a disc diameter ranging from about 0.5 cm to about 5 cm, such as from about 1 cm to about 4 cm, such as from about 2 cm to about 3 cm.

Through selective control over the particular nature of the device and the manner in which it is formed, the resulting device can be effective for sustained release of one or more GLP-1 receptor agonists over a prolonged period of time. For example, the implantable device can release the therapeutic agent(s) for a time period of about 5 days or more, in some embodiments about 10 days or more, in some embodiments from about 21 days or more, and in some embodiments, from about 25 days to about 50 days (e.g., about 30 days). In certain embodiments, the device can release the therapeutic agent(s) for a time period of about 1 month or more, such as about 3 months or more, such as about 6 months or more, such as about 12 months or more, and in some embodiments, from about 12 months to about 36 months. Further, the therapeutic agent(s) can be released in a controlled manner (e.g., zero order or near zero order) over the course of the release time period. After a time period of 21 days, for example, the cumulative release ratio of the implantable device may be from about 10% to about 70%, in some embodiments from about 30% to about 65%, and in some embodiments, from about 40% to about 60%. Likewise, after a time period of 30 days, the cumulative release ratio of the implantable device may still be from about 40% to about 85%, in some embodiments from about 50% to about 80%, and in some embodiments, from about 60% to about 80%. The “cumulative release ratio” may be determined by dividing the amount of the therapeutic agent released at a particulate time interval by the total amount of therapeutic agent initially present, and then multiplying this number by 100. In other embodiments, after a time period of about 55 days, the cumulative release ratio may be less than about 20%, such as less than about 15%. In embodiments, after a time period of about 21 days the cumulative release ratio can be from about 5% to about 15%, such as about 10%.

Of course, the actual dosage level of the GLP-1 receptor agonist delivered will vary depending on the particular GLP-1 receptor agonist employed and the time period for which it is intended to be released. The dosage level is generally high enough to provide a therapeutically effective amount of the GLP-1 receptor agonist to render a desired therapeutic outcome, i.e., a level or amount effective to reduce or alleviate symptoms of the condition for which it is administered. The exact amount necessary will vary, depending on the subject being treated, the age and general condition of the subject to which the GLP-1 receptor agonist is to be delivered, the capacity of the subject's immune system, the degree of effect desired, the severity of the condition being treated, the particular GLP-1 receptor agonist selected and mode of administration of the composition, among other factors. For example, an effective amount will typically range from about 0.001 mg to about 4 mg per day, such as from about 0.01 mg to about 3 mg per day, such as from about 0.1 mg to about 1.5 mg per day of the GLP-1 receptor agonist. In certain embodiments, an effective amount ranges from about 0.5 mg to about 2.5 mg per day, such as from about 1 mg to about 2 mg per day.

The amount of GLP-1 receptor agonist loaded into the device can vary. For example, for implantable devices configured to be inserted and retained for periods of time ranging from about one month but fewer than 12 months, the core can be loaded with 3 mg to about 365 mg of one or more GLP-1 receptor agonist. For implantable devices configured to be inserted and retained for periods of time ranging from about 1 year up to about 3 years, the core can be loaded with about 450 mg to about 900 mg of one or more GLP-1 receptor agonists. Additionally, the amount of GLP-1 receptor agonist loaded into the core can be modified (e.g., increased and/or decreased) depending on the amount of implantation time desired or route of implantation. Additionally, the amount of GLP-1 receptor agonist loaded into the core can be modified based on the use of additional therapeutic agents in addition to the GLP-1 receptor agonists. For example, the amount of GLP-1 receptor agonist loaded into the core can be increased when the implant includes or is co-administered with one or more therapeutic agents known to increase blood glucose levels.

If desired, the device may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the device may be stored. For example, a single film may be folded at one or more points and sealed along its periphery to define the cavity within with the device is located. To use the device, the package may be opened, such as by breaking the seal, and the device may then be removed and implanted into a patient.

### Comparative Example 1

Rod-shaped implants containing liraglutide and ethylene vinyl acetate copolymer (EVA) were produced via hot melt extrusion. The implants contained 40 wt. % Ateva® 4030AC and 60 wt. % liraglutide. EVA was cryogenically ground to a powder and then blended by hand with liraglutide powder. The resulting powder blend was fed into an 11 mm twin-screw extruder. Extrusion was accomplished using a screw speed of 50 rpm with barrel temperatures set to achieve a nominal melt temperature of 65 °C. The extrudate was approximately 3 mm in diameter, and was cut to 1 cm length rods for in vitro drug elution testing.

In vitro drug elution testing was conducted by immersing a single implant in 50 mL of 4 mM phosphate buffered saline solution with a pH of 7.4. Media and implant were placed in an incubator at 37 °C and were gently agitated on a 100 rpm oscillating table. On a periodic basis, elution media was replaced with fresh buffer, and the concentration of liraglutide in the media was measured via HPLC. Exceptionally high sample to sample variability was observed during this test. Several samples exhibited little to no release of liraglutide, whereas others exhibited a rapid burst of drug over the first seven days before drug release completely stopped.

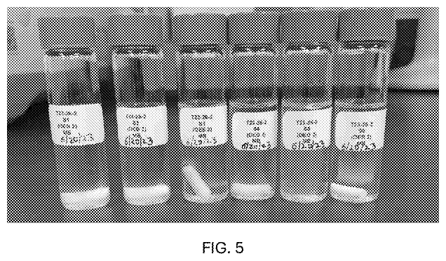

FIG. 5 illustrates visible gel and fibril formation in the drug elution media after seven days.

### Comparative Example 2

To better understand the implications of drug release of liraglutide from a polymeric implant, further investigation was completed.

Liraglutide's physical stability in phosphate buffered saline (PBS) as a function of pH and time was investigated to determine stability effects on the drug release of liraglutide from a polymeric implant. 1 mg/mL solutions of liraglutide were prepared in PBS with pH values ranging from 5.0 to 8.1. Samples were then incubated at 37 °C and monitored for gelation and fibril formation. The times required before gelation/fibril formation were observed are reported in Tables 1-2.

TABLE 1

| pH of buffer | Time to observe gelation/ |
|---|---|
| solution | fibril formation |
| 5.0 | Gelled immediately |
| 5.5 | Gelled immediately |
| 6.0 | 1 day |
| 6.5 | 1 day |
| 7.0 | 1 day |
| 7.4 | 2 days |
| 8.1 | 5 days (fibril formation observed) |

In light of the observations of Table 1, additional stability studies were conducted at pH of 7.4 and 8.1 and the results are shown in Table 2.

TABLE 2

| | | |
|---|---|---|
| Solution | Time to gel | Time to gel |
| concentration | at pH = 7.4 | at pH = 8.1 |

| | | | | | |
|---|---|---|---|---|---|
| 1 | mg/mL | 2 | days | 5 | days |
| 0.5 | mg/mL | 2 | days | 5 | days |
| 0.25 | mg/mL | 2 | days | 6 | days |
| 0.125 | mg/mL | 2 | days | 13 | days |

| | | | |
|---|---|---|---|
| 0.0625 | mg/mL | No gel observed | No gel observed |

| | |
|---|---|
| at 7 days | at 13 days |

### Comparative Example 3

In vitro drug elution testing was conducted as outlined in Comparative Example 1 except by immersing a single implant in 50 ml of 4 mM phosphate buffered saline solution with a pH of 8.1.

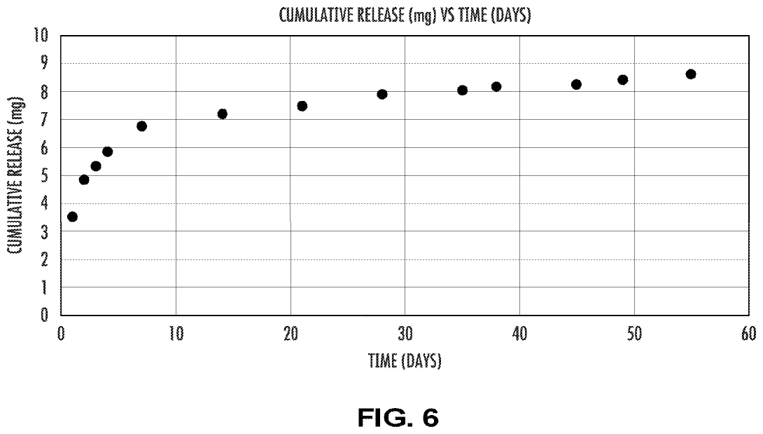

FIG. 6 illustrates the cumulative release (mg) per day of liraglutide for Comparative Example 3 and Table 3 below provides the data points for liraglutide release as shown in FIG. 6. As shown in this Example, drug release was initially robust, but then slowed substantially after approximately seven days.

TABLE 3

| Time (days) | Liraglutide release (mg) |
|---|---|
| 1 | 3.53 |
| 2 | 4.84 |
| 3 | 5.34 |
| 4 | 5.85 |
| 7 | 6.77 |
| 14 | 7.21 |
| 21 | 7.48 |
| 28 | 7.90 |
| 35 | 8.05 |
| 38 | 8.16 |
| 45 | 8.26 |
| 49 | 8.42 |
| 55 | 8.62 |

### Examples 4-24

Compositions containing liraglutide, phosphate buffered saline, and differing excipients were produced. The liraglutide concentrations in the compositions ranged from 6.6 mg/mL to 151 mg/mL. The liraglutide was added in portions to allow for seamless distribution. The phosphate buffered saline had a pH of 7.4 and was diluted 100×. The excipients were added to said composition in an amount ranging from 8 mg/mL to 500 mg/mL. The solutions were stirred in a glass vial with a magnetic stirrer at 100 rpm at ambient temperature. Visual screening was carried out to assess the ability for such excipients to aid in keeping the liraglutide solubilized in the phosphate buffered saline, rather than undergoing phase separation such as gelation.

Table 4 below provides the data for liraglutide gelation as a result of excipient concentration as well as a ratio of suspended liraglutide to excipient.

TABLE 4

| | | | Concentration | |
|---|---|---|---|---|
| | | Concentration | of Excipient | Ratio of |
| | | of Un-Gelled | added to | Liraglutide |
| | | Liraglutide | Liraglutide | to |
| Example # | Chemical Name | (mg/mL) | (mg/mL) | Excipient |
| Example 4 (Representative) | Polyoxyethylene lauryl ether | 21 | 8 | 2.63 |
| Example 5 (Representative) | Polyoxyl 40 Hydrogenated | 27 | 100 | 0.27 |
| | Castor Oil | | | |
| Example 6 (Representative) | Glyco-diosgenin | 148.6 | 94.6 | 1.57 |
| Example 7 (Comparative) | N-decanoyl-N-methylglucamine | 8.9 | 1.07 | 8.32 |
| Example 8 (Representative) | Benzalkonium chloride | 150.8 | 21.4 | 7.05 |
| Example 9 (Representative) | Diethanolamine | 82.5 | 500 | 0.17 |
| Example 10 (Representative) | Glycerol | 43.3 | 500 | 0.09 |
| Example 11 (Comparative) | Labrasol | 9.8 | 100 | 0.10 |
| Example 12 (Representative) | Sodium lauryl sulfate | 145.48 | 20 | 7.27 |
| Example 13 (Comparative) | Mannitol | 10 | 100 | 0.10 |
| Example 14 (Representative) | Glucose | 31.98 | 100 | 0.32 |
| Example 15 (Representative) | Polyethylene glycol 660 | 20.6 | 86.8 | 0.24 |
| | 12-hydoxystearate | | | |
| Example 16 (Representative) | Hydroxypropyl methyl cellulose | 46.125 | 250 | 0.18 |
| Example 17 (Comparative) | D-alpha-tocopherol polyethylene | 12.46 | 11.96 | 1.04 |
| | glycol 1000 succinate | | | |
| Example 18 (Comparative) | Polysorbate 80 | 14.62 | 50 | 0.29 |
| Example 19 (Comparative) | Polysorbate 20 | 8.12 | 50 | 0.16 |
| Example 20 (Comparative) | Polyethylene glycol 6000 | 6.6 | 250 | 0.03 |
| Example 21 (Comparative) | Poloxamer 124 | 5.86 | 100 | 0.06 |
| Example 22 (Representative) | Hydroxypropyl beta cyclodextrin | 25.6 | 222.22 | 0.12 |
| Example 23 (Comparative) | PropyleneGlycol | 10.18 | 200 | 0.05 |
| Example 24 (Comparative) | Kollidon 30 | 11.5 | 59.4 | 0.19 |

As shown in Table 4, inclusion of certain excipients, as evidenced by the representative examples, resulted in compositions where less therapeutic agent fell out of suspension or gelled. The liraglutide concentrations that remain un-gelled varied from 6.6 mg/mL to 150.8 mg/mL. Accordingly, these mixtures including the excipients and therapeutic agents can facilitate higher drug loading when formulated with the core of an implant as described herein.

### Examples 25-26

Vital dose Ethylene Vinyl acetate (40% VA) was extruded with Liraglutide and solubility enhancing excipients to yield implants using a hot melt extruder. Formulations with these solubility enhancing excipient are presented in Table 5. Drug release study was initiated with Phosphate Buffered Saline maintained at pH 7.4 in incubators maintained at 37 °C and stirring at 100 rpm. Liraglutide is prone to gelling when extruded with EVA. The addition of solubility enhancing excipients helps in enhancing physical stability of Liraglutide.

TABLE 5

| | Example 25 | Example 26 |
|---|---|---|
| | (w/w) | (w/w) |
| Liraglutide | 10% | 36% |
| Hydroxypropyl Betacyclodextrin (HPBCD) | 50% | — |
| Glyco-diosgenin (GDN) | — | 24% |
| Ethylene Vinyl Acetate (EVA 40% VA) | 40% | 40% |

### Preparation

A powder blend of Liraglutide and HPBCD or Liraglutide and GDN in the ratio as indicated in Table 5 was prepared in the buffer. The buffer used was PBS, pH 7.4 diluted 100× by purified water. Firstly, excipient either HPBCD or GDN was dissolved in buffer followed by addition of Liraglutide under constant stirring. After complete dissolution of liraglutide the solution was put under vacuum to remove the buffer. A dry powder blend of Liraglutide and the excipient was prepared under the vacuum. This powder blend was mixed with EVA (40% VA) in the ratio as indicated in Table 5. This final powder blend with EVA included was then extruded using a hot melt extruder within temperature range of 60-70 °C to yield implant rod cores.

### Membrane Application

Membranes were applied on implant cores to control the drug release. The membrane compositions with mannitol average particle sizes are shown in Table 6.

TABLE 6
Membrane compositions

| Ingredients | Membrane 1 | Membrane 2 | Membrane 3 |
|---|---|---|---|
| Mannitol (%) | 20% | 45% | 45% |
| Ethylene Vinyl Acetate | 80% | 55% | 55% |
| (40% VA) | | | |
| Mannitol Average particle | 125 μm | 25 μm | 125 μm |
| size (μm) | | | |

The ingredients were blended in the composition as shown in Table 3. The blends were extruded using hot melt extrusion. These extruded membrane materials were then applied on the implant cores using vacuum compression molding (VCM) technique.

Each implant with membranes had a length of 14-19 mm and a diameter of ~2.5 mm.

### Elution Studies

Drug release study was initiated by adding implant rods into Phosphate Buffered Saline maintained at pH 7.4. These samples in PBS were run in incubators maintained at 37 °C and equipped with a table oscillating at 100 rpm.

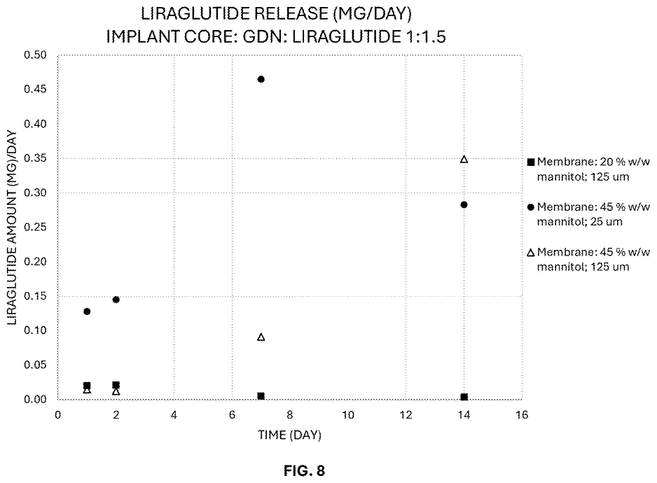

Drug release data through day 14th are illustrated in FIGS. 8-12 and Tables 7-12.

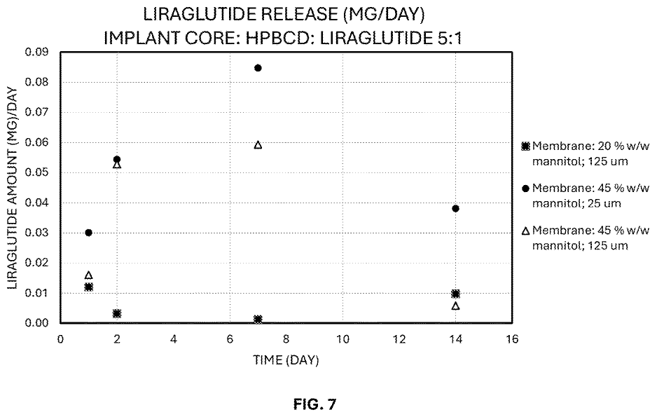

FIGS. 7-8 and Tables 7-8 illustrate liraglutide release (mg/day) from implants in Examples 25-26.

TABLE 7

| | Amount (mg)/Time (day) | | |
|---|---|---|---|
| | Implant Core: | Implant Core: | Implant Core: |
| | HPBCD:Liraglutide | HPBCD:Liraglutide | HPBCD:Liraglutide |
| | 5:1 | 5:1 | 5:1 |
| | Implant Membrane: | Implant Membrane: | Implant Membrane: |
| Time | 0.25 mm thick; 20% | 0.25 mm thick; 20% | 0.25 mm thick; 20% |
| (days) | mannitol; 125 μm | mannitol; 125 μm | mannitol; 125 μm |
| 1 | 0.01 | 0.03 | 0.02 |
| 2 | 0.00 | 0.05 | 0.05 |
| 7 | 0.00 | 0.08 | 0.06 |
| 14 | 0.01 | 0.04 | 0.01 |

TABLE 8

| | Amount (mg)/Time (day) | | |
|---|---|---|---|
| | Implant Core: | Implant Core: | Implant Core: |
| | GDN:Liraglutide | GDN:Liraglutide | GDN:Liraglutide |
| | 1:1.5 | 1:1.5 | 1:1.5 |
| | Implant Membrane: | Implant Membrane: | Implant Membrane: |
| Time | 0.25 mm thick; 20% | 0.25 mm thick; 20% | 0.25 mm thick; 20% |
| (days) | mannitol; 125 μm | mannitol; 125 μm | mannitol; 125 μm |
| 1 | 0.02 | 0.13 | 0.01 |
| 2 | 0.02 | 0.15 | 0.01 |
| 7 | 0.01 | 0.46 | 0.09 |
| 14 | 0.00 | 0.28 | 0.35 |

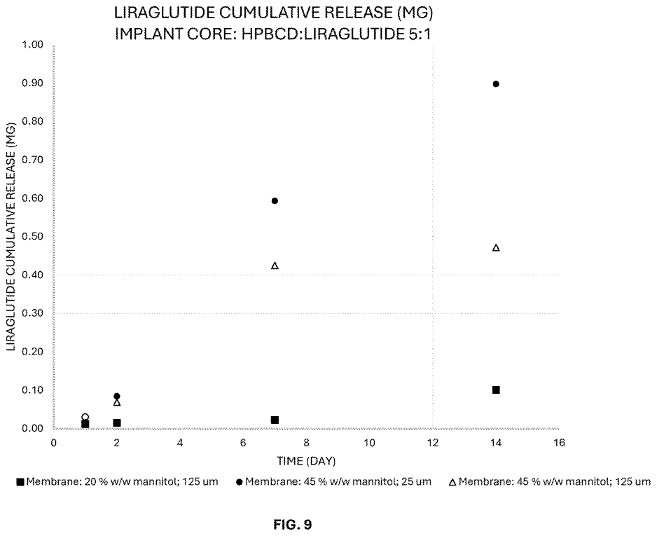

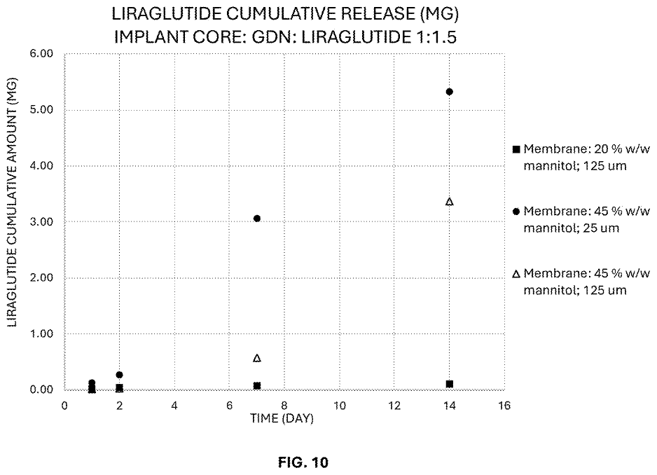

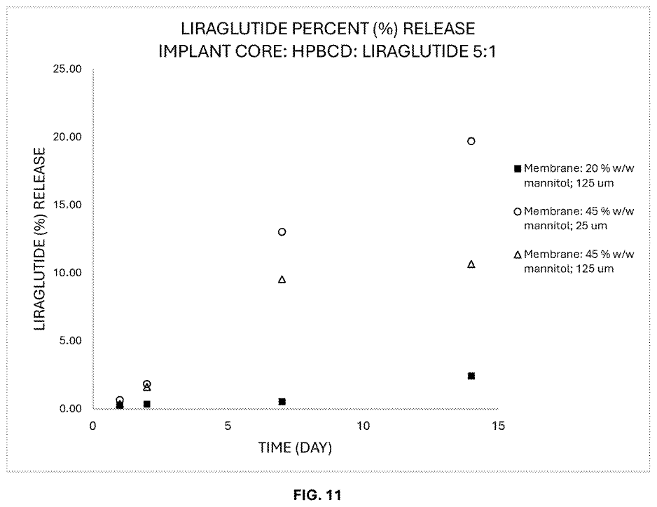

FIGS. 9-10 and Tables 9-10 illustrate cumulative release of liraglutide from implants in Examples 25-26.

TABLE 9

| | Cumulative amount (mg) release | | |
|---|---|---|---|
| | Implant Core: | Implant Core: | Implant Core: |
| | HPBCD:Liraglutide | HPBCD:Liraglutide | HPBCD:Liraglutide |
| | 5:1 | 5:1 | 5:1 |
| | Implant Membrane: | Implant Membrane: | Implant Membrane: |
| Time | 0.25 mm thick; 20% | 0.25 mm thick; 20% | 0.25 mm thick; 20% |
| (days) | mannitol; 125 μm | mannitol; 125 μm | mannitol; 125 μm |
| 1 | 0.01 | 0.03 | 0.02 |
| 2 | 0.02 | 0.08 | 0.07 |
| 7 | 0.02 | 0.59 | 0.43 |
| 14 | 0.10 | 0.90 | 0.47 |

TABLE 10

| | Cumulative amount (mg) release | | |
|---|---|---|---|
| | Implant Core: | Implant Core: | Implant Core: |
| | GDN:Liraglutide | GDN:Liraglutide | GDN:Liraglutide |
| | 1:1.5 | 1:1.5 | 1:1.5 |
| | Implant Membrane: | Implant Membrane: | Implant Membrane: |
| Time | 0.25 mm thick; 20% | 0.25 mm thick; 20% | 0.25 mm thick; 20% |
| (days) | mannitol; 125 μm | mannitol; 125 μm | mannitol; 125 μm |
| 1 | 0.02 | 0.13 | 0.01 |
| 2 | 0.04 | 0.27 | 0.03 |
| 7 | 0.08 | 3.06 | 0.57 |
| 14 | 0.11 | 5.33 | 3.37 |

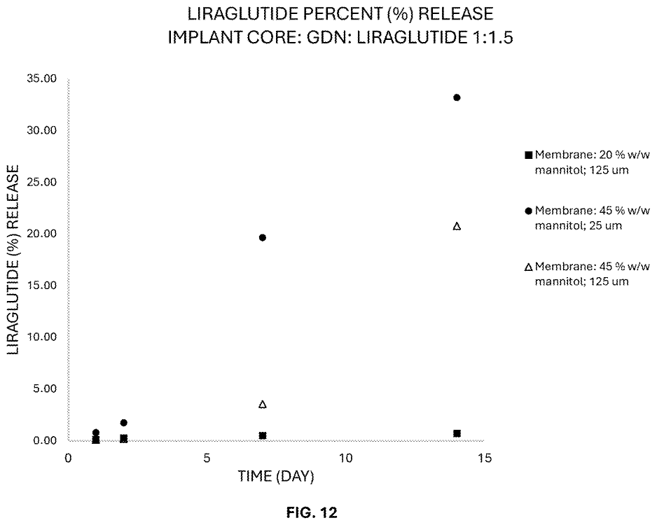

FIGS. 11-12 and Tables 11-12 illustrate percentage of liraglutide released from implants in Examples 25-26.

TABLE 11

| | Percent (%) release | | |
|---|---|---|---|
| | Implant Core: | Implant Core: | Implant Core: |
| | HPBCD:Liraglutide | HPBCD:Liraglutide | HPBCD:Liraglutide |
| | 5:1 | 5:1 | 5:1 |
| | Implant Membrane: | Implant Membrane: | Implant Membrane: |
| Time | 0.25 mm thick; 20% | 0.25 mm thick; 20% | 0.25 mm thick; 20% |
| (days) | mannitol; 125 μm | mannitol; 125 μm | mannitol; 125 μm |
| 1 | 0.29 | 0.65 | 0.38 |
| 2 | 0.36 | 1.84 | 1.61 |
| 7 | 0.54 | 13.03 | 9.56 |
| 14 | 2.43 | 19.71 | 10.67 |

TABLE 12

| | Percent (%) release | | |
|---|---|---|---|
| | Implant Core: | Implant Core: | Implant Core: |
| | GDN:Liraglutide | GDN:Liraglutide | GDN:Liraglutide |
| | 1:1.5 | 1:1.5 | 1:1.5 |
| | Implant Membrane: | Implant Membrane: | Implant Membrane: |
| Time | 0.25 mm thick; 20% | 0.25 mm thick; 20% | 0.25 mm thick; 20% |
| (days) | mannitol; 125 μm | mannitol; 125 μm | mannitol; 125 μm |
| 1 | 0.14 | 0.79 | 0.09 |
| 2 | 0.28 | 1.73 | 0.17 |
| 7 | 0.49 | 19.65 | 3.55 |
| 14 | 0.71 | 33.21 | 20.79 |

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. An implantable device for delivery of a therapeutic agent, the device comprising:
a core comprising a core polymer matrix within which is dispersed at least one therapeutic agent comprising one or more glucagon-like peptide-1 (GLP-1) receptor agonists and at least one excipient,
wherein the ratio of the one or more glucagon-like peptide-1 (GLP-1) receptor agonists to the at least one excipient ranges from about 20:1 to about 1:20, and
wherein the core polymer matrix includes an ethylene vinyl acetate copolymer.

2. The implantable device of claim 1, wherein the ethylene vinyl acetate copolymer has a vinyl acetate monomer content of from about 10 wt. % to about 50 wt. % of the copolymer.

3. The implantable device of claim 1, wherein the GLP-1 receptor agonist has a molecular weight of about 5,000 Da or less.

4. The implantable device of claim 1, wherein the GLP-1 receptor agonist comprises dulaglutide, exenatide, semaglutide, liraglutide, lixisenatide, tirzepatide, or combinations thereof.

5. The implantable device of claim 1, wherein a combination of the one or more glucagon-like peptide-1 (GLP-1) receptor agonists and the at least one excipient comprises from about 5 wt. % to about 95 wt. % of the core.

6. The implantable device of claim 1, wherein the excipient comprises anon-ionic surfactant, a cationic surfactant, an anionic surfactant, a sugar or sugar derivative, a polymer, a glycol, or combinations thereof.

7. The implantable device of claim 6, wherein the cationic surfactant comprises benzalkonium chloride; the anionic surfactant comprises sodium lauryl sulfate; and the non-ionic surfactant comprises polyoxyethylene laurel ether, polyoxyl 40 hydrogenated castor oil, diethanolamine, polyethylene glycol 660 12-hydoxystearate, D-alpha-tocopherol polyethylene glycol 1000 succinate, Polysorbate 80, N-Decanoyl-N-methylglucamine, capyrlocaproyl polyoxyl-8 glycerides, glyco-diosgenin or a combination thereof, including derivatives thereof.

8. The implantable device of claim 6, wherein the sugar or sugar derivative comprises glycerol, mannitol, glucose, beta cyclodextrin, hydroxypropyl beta cyclodextrin, or a combination thereof.

9. The implantable device of claim 8, wherein the sugar or sugar derivative comprises mannitol having an average particle size of at least about 25 μm and less than about 125 μm.

10. The implantable device of claim 6, wherein the polymer comprises hydroxylpropyl methyl cellulose, Kollidon 30, or a combination thereof.

11. The implantable device of claim 6, wherein the glycol comprises polypropylene glycol.

12. The implantable device of claim 1, wherein the core comprises from about 5 wt. % to about 70 wt. % of the one or more GLP-1 receptor agonists.

13. The implantable device of claim 1, wherein the core comprises from about 5 wt. % to about 60 wt. % of the one or more excipients.

14. The implantable device of claim 1, wherein the one or more GLP-1 receptor agonists are released from the device in an amount sufficient to deliver from about 0.001 mg of GLP-1 receptor agonist to about 4 mg of GLP-1 receptor agonist per day for a time period of at least one month.

15. The implantable device of claim 14, wherein the one or more GLP-1 receptor agonists are released from the device in an amount sufficient to deliver from about 0.01 mg of GLP-1 receptor agonist to about 1.5 mg of GLP-1 receptor agonist per day for a time period of at least one month.

16. The implantable device of claim 1, comprising a membrane layer disposed on an outer surface of the core, the membrane layer including a membrane polymer matrix containing an ethylene vinyl acetate copolymer.

17. The implantable device of claim 1, wherein after a time period of about 21 days the cumulative release ratio of the implantable device is from about 10% to about 70%.

18. A method for prohibiting and/or treating a condition, disease, and/or cosmetic state of a patient, the method comprising subcutaneously implanting the implantable device of claim 1 in the patient.

19. A method of preparing the implantable device of claim 1 comprising:
mixing the at least one therapeutic agent, the at least one excipient, and at least one solvent to form a drug mixture, wherein the therapeutic agent has a concentration of at least about 0.001 mg/mL to about 300 mg/mL,
drying the drug mixture to remove the at least one solvent forming a dried drug mixture, and
melt-extruding the dried drug mixture with the core polymer matrix to form the implantable device.

20. The method of claim 19, wherein the weight ratio of therapeutic agent to the excipient in the dried drug mixture is from about 20:1 to about 1:20.
